(19) United States
(12) Patent Application Publication
(10) Pub. No.: US 2010/0068238 A1
(43) Pub. Date: Mar. 18, 2010

(54) Implantable Medical Devices Comprising a Flavonoid or Derivative Thereof for Prevention of Restenosis

(76) Inventor: Nandkishore Managoli, Gujarat (IN)

Correspondence Address: THE NATH LAW GROUP, 112 South West Street, Alexandria, VA, 22314, US

(21) Appl. No.: 11/988,805

(22) PCT Filed: Jun. 26, 2006
(86) PCT No.: PCT/IB2006/001848

§ 371 Date: Mar. 23, 2009

Related U.S. Application Data
(60) Provisional application No. 60/699,509, filed on Jul. 15, 2005.

(30) Foreign Application Priority Data
Jul. 15, 2005 (EP) 05106528.2

(51) Int. Cl. A61F 2/82; A61F 2/04; A61K 31/4355; A61P 9/00

(52) U.S. Cl. 424/423; 514/456; 514/291

(57) ABSTRACT

The present invention relates to implantable medical devices, such as stents, that comprise a composition for controlled delivery of flavonoids or a derivative thereof. The flavonoids are aimed at preventing or reducing secondary complications which can occur following implantation of the device such as e.g. occlusive and catastrophic vascular phenomena. The invention further relates to the inclusion of additional therapeutic agents in the system that may have antiproliferative, antimitotic, antimicrobial, anticoagulant, fibrinolytic, anti-inflammatory, immunosurpressive, and anti-angiogenic activities. The composition comprising the flavanoids and optional further therapeutic agents may be used in methods for treating or preventing narrowing or obstruction of the body passageway. In particular the devices and compositions of the invention are useful in methods for treating or preventing restenosis, e.g. subsequent to angioplasty and/or for preventing or reducing acute, subacute and chronic secondary complications associated with angioplasty, such as e.g. thrombus.

### FIELD OF THE INVENTION

The present invention relates to implantable medical devices, such as stents, that comprise a system for controlled delivery of therapeutic agents. In particular the invention relates to the inclusion of additional agents in the system that are aimed at preventing or reducing secondary complications which can occur following implantation of the device such as e.g. occlusive and catastrophic vascular phenomena.

### BACKGROUND OF THE INVENTION

The human or animal body comprises many passageways for transport of essential materials. These include e.g. the vascular system for transport of blood, the various passageways of the gastrointestinal tract, the urinary tract, the airways, as well as the reproductive tracts. Various insults to these passageways (injury, surgical procedures, inflammation or neoplasms) can produce narrowing or even obstruction of such body passageways, with serious consequences that may ultimately result in death.

One approach to the problem of narrowing or obstructed body passageways has been the insertion of endoluminal stents. Briefly, stents are devices having a generally tubular structure that are placed into the lumen of a body passageway to physically hold open a passageway that is narrowed or blocked by e.g. a tumor or other tissues/substances/pathological processes like occlusive atherothrombosis. A major problem is however that frequently the body responds to the implanted stent by ingrowth into the lumen of the stent, thereby again narrowing or blocking the passageway into which the stent was placed. E.g. in the case of stents that are used in the context of a neoplastic obstruction, the tumor is usually able to grow into the lumen of the stent. Also in non-neoplastic settings the presence of a stent in the lumen of a body passageway can induce the ingrowth of reactive or inflammatory tissue (e.g., blood vessels, fibroblasts and white blood cells) into lumen of the stent. Particularly in a vascular disease setting restenosis subsequent to balloon angioplasty (with or without stenting) is a major problem. Multiple processes, including thrombosis, inflammation, growth factor and cytokine release, cell proliferation, cell migration and extracellular matrix synthesis each contribute to the restenotic process. Upon pressure expansion of an intracoronary balloon catheter during angioplasty, both endothelial and smooth muscle cells within the vessel wall become injured, initiating proliferative, thrombotic and inflammatory responses that ultimately can lead to occlusion of the implanted stent.

Various anti-proliferative and anti-angiogenic agents have been suggested for prevention of restenosis, including e.g. heparin, taxol, methotraxate, colchicine, vincristine, vinblastine and rapamycin. Although the systemic use of some of these agents showed some success in animal models, the dosages required effective required in these experiments is too high for systemic use in humans. Therefore, in situ, or site-specific drug delivery using stents coated with controlled-release formulation for anti-restenotic agents have been developed (see e.g. WO 90/13332; WO 91/12779; EP 0 551 182; and EP 0 706 376).

However, despite the success of such anti-restenotic drug-eluting stents in reducing restenosis, reports have issued that in a significant number of cases the implanted stents are responsible for secondary complications such as e.g. sub acute thrombus (SAT), as well as, late thrombus and associated mortality (see e.g. Liistro and Colombo, 2001, Heart 86: 262-4; Cutlip et al., 2001, Circulation 103: 1967-71). There is thus still a need in the art for implantable medical devices for controlled sustained delivery of therapeutic agents that reduce or prevent the secondary complications that may occur after implantation of the device.

### DESCRIPTION OF THE INVENTION

The present invention relates to implantable medical devices comprising or coated with preferably multiple layers of therapeutic agents and preferably biodegradable polymers to create a novel, controlled drug delivery system useful in managing catastrophic occlusive phenomena and also in preventing further secondary complications which can occur following implantation of the device. The novelty of this invention lies in the strategic combination of more than one therapeutic agent each in definitive dosages and coated with biodegradable polymer to ensure optimum release in a controlled manner. This invention also relates to the multifunctionality of the drug delivery system, which, owing to the strategic combination of therapeutic agents, each with multiple functions and the ability of the drug delivery system to release the therapeutic agents in a controlled manner, provides an efficient and safe system to manage catastrophic occlusive conditions and also prevent the secondary complications that can occur after implanting the device, particularly in the context of vascular angioplasty. The invention also relates to the usage of biodegradable polymers, which ensure that the therapeutic agents and the polymer cease to exist in the vessel wall after the predetermined period of 48-55 days after implantation. In the context of vascular angioplasty the devices of the invention thus deprive the vascular elements a nidus to initiate a cascade of detrimental secondary effects.

Angioplasty may be performed as part of “revascularization” treatment for “artherosclerosis”, which is herein understood to mean disease in which plaque, made up of cholesterol, fats, calcium, and scar tissue, builds up in the wall of blood vessels, narrowing the lumen and interfering with blood flow. “Revascularization”, herein means any treatment that re-establishes brisk blood flow through a narrowed artery, including bypass surgery, angioplasty, stenting, and other interventional procedures. Secondary complications following revascularisation may include restenosis, neointima, neointimal hyperplasia and thrombosis. “Restenosis” is herein defined as the re-narrowing of an artery in the same location of a previous treatment; clinical restenosis is the manifestation of an ischemic event, usually in the form of recurrent angina. “Neointima” is herein defined as the scar tissue made up of cells and cell secretions that often forms as a result of vessel injury following angioplasty or stent placement as part of the natural healing process. “Neointimal hyperplasia” herein means excessive growth of smooth muscle cells from the inner lining of the artery. After angioplasty and/or stenting, excessive growth of these cells can narrow the artery again. Thrombosis herein means the formation of a blood clot within a blood vessel or the heart cavity itself and a “thrombus” is a blood clot.

Three pathophysiological phases can be distinguished subsequent to revascularization. Stage I, the thrombotic phase (days 0-3 after revascularization). This stage consists of rapid thrombus formation. The initial response to arterial injury is explosive activation, adhesion, aggregation, and platelet deposition. The platelet thrombus may frequently be large and can grow large enough to occlude the vessel, as occurs in myocardial infarction. Within 24 hours, fibrin-rich thrombus accumulates around the platelet site. Two morphologic features are prominent: 1) platelet/fibrin, and 2) fibrin/red cell thrombus. The platelets are densely clumped at the injury site, with the fibrin/red cell thrombus attached to the platelet mass.

Stage II, the recruitment phase (days 3-8). The thrombus at arterial injury sites develops an endothelial cell layer. It is unclear whether the cells are truly endothelial cells despite their histopathologic appearance. Shortly after the endothelial cells appear, an intense cellular infiltration occurs. The infiltration is principally monocytes that become macrophages as they leave the bloodstream and migrate into the subendothelial mural thrombus. Lymphocytes also are present, and both types of cells demarginate from the bloodstream. This infiltrate develops from the luminal side of the injured artery, and the cells migrate progressively deeper into the mural thrombus.

Stage III, the proliferative phase: (day 8 to final healing). Actin-positive cells colonize the residual thrombus from the lumen, forming a “cap” across the top of the mural thrombus in this final stage. The cells progressively proliferate toward the injured media, resorbing thrombus until it is completely gone and replaced by neointimal cells. At this time the healing is complete. In the pig this process requires 21-40 days, depending on residual thrombus thickness. Smooth muscle cell migration and proliferation into the degenerated thrombus increases neointimal volume, appearing greater than that of thrombus alone. The smooth muscle cells migrate from sites distant to the injury location, and the resorbing thrombus becomes a bioabsorbable “proliferation matrix” for neointimal cells to migrate and replicate. The thrombus is colonized at progressively deeper levels until neointimal healing is complete.

One object of the invention is thus to provide for compositions that may be used in methods to prevent or reduce secondary complications following revascularisation, which may include restenosis, neointima, neointimal hyperplasia and thrombosis. In a first aspect, therefore, the present invention relates to an implantable medical device that comprises a composition for controlled release of a flavonoid or a derivative thereof. Flavonoids are polyphenolic substances based on a flavan nucleus, comprising 15 carbon atoms, arranged in three rings as C₆—C₃—C₆ with a general structure according to formula I:

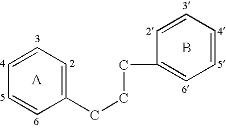

The chemical structure of flavonoids are based on a C₁₅ skeleton with a chromane ring bearing a second aromatic ring B in position 2, 3 or 4 (formula II). In a few cases, the six-membered heterocyclic ring C occurs in an isomeric open form or is replaced by a five-membered ring.

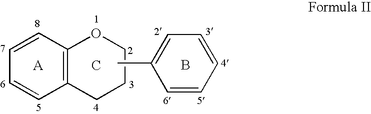

Flavonoids are biosynthetically derived from acetate and shikimate such that the A ring has a characteristic hydroxylation pattern at the 5 and 7 position. The B ring is usually 4′,3′4′, or 3′4′5′-hydroxylated. Flavonoids have generally been classified into 12 different subclasses by the state of oxidation and the substitution pattern at the C2-C3 unit. There are a number of chemical variations of the flavonoids, such as, the state of oxidation of the bond between the C2-C3 position and the degree of hydroxylation, methoxylation or glycosylation (or other substituent moieties) in the A, B and C rings and the presence or absence of a carbonyl at position 4. Flavonoids for use in the present invention include, but are not limited to, members of the following subclasses: chalcone, dihydrochalcone, flavanone, flavonol, dihydroflavonol, flavone (found in citrus fruits), flavanol, isoflavone, neoflavone, aurone, anthocyanidin (found in cherries, strawberries, grapes and colored fruits), proanthocyanidin (flavan-3,4-diol) and isoflavane. Thus far, more than 10,000 flavonoids have been identified from natural sources. Berhow (1998) pp. 67-84 in Flavonoids in the Living System, ed. Manthey et al., Plenum Press, NY.

Flavonoids have a number of activities that are useful in the context of the present invention. These activities include e.g. anti-platelet aggregation, anti-thrombotic, anti-inflammatory, anti-atherogenic, anti-oxidant, inhibition of angiogenesis, inhibition of lipid oxidation and peroxidation, lipid-lowering and inhibition of cell cycle. Preferably, a composition of the present invention at least comprises a flavonoid with anti-platelet aggregation activity and/or anti-thrombotic activities. These activities may be assayed by methods know to the skilled person per se (see e.g. E. M. Van Cott, M.D., and M. Laposata, M. D., Ph.D., “Coagulation.” In: Jacobs D S et al, ed. “The Laboratory Test Handbook”, 5th Edition. Lexi-Comp, Cleveland, 2001; 327-358). A composition of the invention may however comprises more than one flavonoid. Preferably in that case at least one flavonoid comprises anti-platelet aggregation activity and/or anti-thrombotic activities and the other flavonoid(s) comprise other useful activities as indicated above.

A preferred flavonoid for use in the compositions of the present invention is a flavonoid that mediates the above anti-platelet aggregation, anti-thrombotic and anti-inflammatory activities through their ability to inhibit DNA topoisomerase II, protein tyrosine kinases, and/or nitric oxide synthase and/or modulation of the activity of NF-kappaB. These activities may be assayed by methods known to the skilled person per se (see e.g. Andrea et al., 1991, Mol. Pharmacol. 40:495-501; the HitHunter EFC-TK assay from DiscoverX, Fremont, Calif.; Webb and Ebeler, 2004, Biochem. J. 384: 527-41; Akiyama et al., 1987, J. Biol. Chem., Vol. 262, 5592-95).

Further properties of the flavonoids that are relevant in the context of the present invention include: inhibition of cell cycle, inhibition of smooth muscle cell proliferation and/or migration. A flavonoid preferably is capable of exerting the above activities when used singly. However, the above properties of the flavonoid may be further enhance by exploiting the synergy between the flavonoid and further therapeutic agents (as listed below herein), in particular paclitaxel, sirolimus and/or rapamicin.

A flavonoid for use in the compositions of the present invention may be selected from narigenin, naringin, eriodictyol, hesperetin, hesperidin (esperidine), kampferol, quercetin, rutin, cyanidol, meciadonol, catechin, epi-gallocatechin-gallate, taxifolin (dihydroquercetin), genistein, genistin, daidzein, biochanin, glycitein, chrysin, diosmin, luetolin, apigenin, tangeritin and nobiletin. A preferred flavonoid for use in the compostions of the present invention is a flavanone, a flavonol, or an isoflavone. More preferably the flavonoid is selected from genistein, quercetin, rutin, narigenin and naringin. Alternatively, a mixture of flavonoids extracted from plant-material may be used in the composition of the invention such as e.g. extracts from grapes (Vitis vinifera), in particular grape seed or grape skin (see e.g. Shanmuganayagam et al., 2002, J. Nutr. 132:3592-98). Furthermore, derivatives of the above flavonoids may be used in the compositions of the invention. By “derivative” is meant a compound derived from and thus non-identical to another compound. As used herein, a derivative shares at least one function with the compound from which it is derived, but differs from that compound structurally. Derivatives of flavonoids include without limitation those that differ from flavonoids due to modifications (including without limitation substitutions, additions and deletions) in a ring structure or side chain. Derivatives of flavonoids include those compounds which differ from flavonoids in structure. These structural differences can be, as non-limiting examples, by addition, substitution or re-arrangement of hydroxyl, acyl or other group. As a non-limiting example, a flavonoids derivative can have additional (substituted or non-substituted) alkyl groups attached. In addition, flavonoids derivatives include compounds which have been conjugated to another chemical moiety, such as a sugar or other carbohydrate. Derivatives also include salts of flavonoids.

A particularly preferred flavonoid for use in the compositions of the present invention is genistein or an analogue of genistein. Genistein is the aglycone (aglucon) of genistin. The isoflavone is found naturally as the glycoside genistin and as the glycosides 6″-O-malonylgenistin and 6″-O-acetylgenistin. Genistein and its glycosides are mainly found in legumes, such as soybeans and chickpeas. Genistein is a solid substance that is practically insoluble in water. Its molecular formula is C₁₅H₁₀O₅, and its molecular weight is 270.24 daltons. Genistein is also known as 5,7-dihydroxy-3-(4-hydroxyphenyl)-4H-1-benzopyran-4-one, and 4′,5,7-trihydroxyisoflavone. Genistin, which is the 7-beta glucoside of genistein, has greater water solubility than genistein. Genistein has the following structural formula:

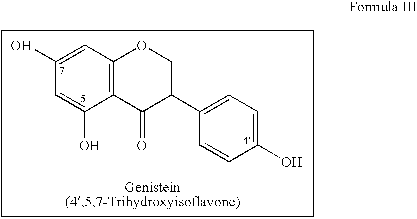

Genistein has been found to have a number of antioxidant activities. It is a scavenger of reactive oxygen species and inhibits lipid peroxidation. It also inhibits superoxide anion generation by the enzyme xanthine oxidase. In addition, genistein, in animal experiments, has been found to increase the activities of the antioxidant enzymes superoxide dismutase, glutathione peroxidase, catalase and glutathione reductase. Genistein's activities include upregulation of apoptosis, inhibition of angiogenesis, inhibition of platelet aggregation, inhibition of DNA topoisomerase II and inhibition of protein tyrosine kinases. Genistein has been resported to have anti-carcinogenic activity, anti-atherogenic activity, lipid-lowering activity, and it may help protect against osteoporosis. A genistein or an analogue thereof for use in the present invention preferably is an inhibitor of tyrosine kinases (as may be assayed as indicated above). Alternatively, other tyrosine kinase inhibitors may be used instead of genistein in the context of the invention, including e.g. erbstatin, herbamycin A, lavendustine-c and hydroxycinnamates. A genistein or an analogue thereof for use in the present invention preferably is an DNA topoisomerase II inhibitor (as may be assayed as indicated above). A genistein or an analogue thereof for use in the present invention preferably is an inhibitor of platelet aggregation and therefore, an inhibitor of thrombus formation (as may be assayed as indicated above). Further properties of genistein or its analogues that are relevant in the context of the present invention include: inhibition of cell cycle, inhibition of smooth muscle cell proliferation and/or migration.

Genistein and/or its analogues are preferably capable of exerting the above activities when used singly. However, the above properties of genistein and/or its analogues may be further enhance by exploiting the synergy between genistein and/or its analogues and further therapeutic agents (as listed herein below), in particular paclitaxel, sirolimus and/or rapamicin. Analogues of genistein include genistin and daidzein.

Another particularly preferred flavonoid for use in the compositions of the present invention is quercetin or an analogue of quercetin. Quercetin is typically found in plants as glycone or carbohydrate conjugates. Quercetin itself is an aglycone or aglucon. That is, quercetin does not possess a carbohydrate moiety in its structure.

Analogues of quercetin include its glycone conjugates include rutin and thujin. Rutin is also known as quercetin-3-rutinoside. Thujin is also known as quercitrin, quercetin-3-L-rhamnoside, and 3-rhamnosylquercetin. Onions contain conjugates of quercetin and the carbohydrate isorhamnetin, including quercetin-3,4′-di-O-beta glucoside, isorhamnetin-4′-O-beta-glucoside and quercetin-4′-O-beta-glucoside. Quercetin itself is practically insoluble in water. The quercetin carbohydrate conjugates have much greater water solubility then quercetin.

Quercetin is known chemically as 2-(3,4-dihydroxyphenyl)-3,5,7-trihydroxy-4H-1-benzopyran-4-one and 3,3′,4′5,7-pentahydroxy flavone. It is also known as meletin and sophretin and is represented by the following structural formula:

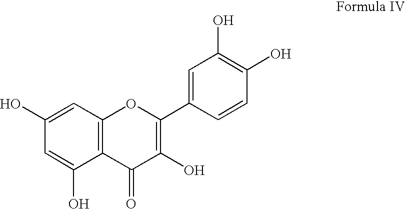

Quercetin is a phenolic antioxidant and has been shown to inhibit lipid peroxidation. In vitro and animal studies have shown that quercetin inhibits degranulation of mast cells, basophils and neutrophils. Such activity account, in part, for quercetin's anti-inflammatory and immunomodulating activities. Other in vitro and animal studies show that quercetin inhibits tyrosine kinase and nitric oxide synthase and that it modulates the activity of the inflammatory mediator, NF-kappaB. Further activities of quercetin include anti-viral and anti-cancer activity. Quercetin is further known to inhibit aldose reductase. A quercetin or an analogue thereof for use in the present invention preferably is an inhibitor of tyrosine kinases (as may be assayed as indicated above). Alternatively, other tyrosine kinase inhibitors (indicated above) may be used instead of quercetin in the context of the invention (as may be assayed as indicated above). A quercetin or an analogue thereof for use in the present invention preferably is an nitric oxide synthase inhibitor (as may be assayed as indicated above). A quercetin or an analogue thereof for use in the present invention preferably is an inhibitor of platelet aggregation and therefore, an inhibitor of thrombus formation (as may be assayed as indicated above).

Further properties of quercetin or its analogues that are relevant in the context of the present invention include: inhibition of cell cycle, inhibition of smooth muscle cell proliferation and/or migration. Suitable analogues/derivatives of quercetin include its glycone conjugates rutin and thujin.

Quercetin and/or its analogues are preferably capable of exerting the above activities when used singly. However, the above properties of quercetin and/or its analogues may be further enhance by exploiting the synergy between quercetin and/or its analogues and further therapeutic agents (as listed herein below), in particular paclitaxel, and/or sirolimus (rapamicin).

The dosage or concentration of a flavonoid or derivative thereof based on surface area on a stent (e.g. a typical coronal stent) may be is 0.1 and 40 μg/mm². Preferably the dosage of a flavonoid or derivative thereof based on surface area of a device of the invention is more than about 0.2., 0.5, 1.0, 2.0, 5.0 or 10 μg/mm². Preferably the dosage of a flavonoid or derivative thereof based on surface area of a device of the invention is less than about 30.0, 20.0, 15.0, 10.0, 5.0, 3.0 or 2.0 μg/mm² Consequently, the amount of the flavonoid or derivative thereof will increase linearly with the length of the stent. E.g. for a typical series of coronary stent varying in length from 8.00 to 39.00 mm, the total flavonoid (or derivative thereof) content will vary from 28 μg to 3500 μg.

The composition for controlled release comprised in a device according to the invention preferably comprises a soluble polymer. As used herein, a soluble polymeric material is a material that has water solubility such that upon exposure to a body fluid an amount of the material will dissolve or erode over time (a period of several days, weeks or even months). “Body fluid” here refers to fluids in the body of a mammal including, but not limited to, blood, urine, saliva, lymph, plasma, gastric, biliary, or intestinal fluids, seminal fluids, and mucosal fluids or humors. A degradable material is a material that can decompose, degenerate, degrade, depolymerize, or otherwise reduce the molecular weight of the starting compound(s) such that the resulting compound(s) is soluble in water or, if insoluble, can be suspended in a body fluid and transported away from the implantation site without clogging the flow of the body fluid. A preferred resorbable polymer is a polymer that is soluble, degradable as defined above, or is an aggregate of soluble and/or degradable material(s) with insoluble material(s) such that, with the resorption of the soluble and/or degradable materials, the residual insoluble materials are of sufficiently fine size such that they can be suspended in a body fluid and transported away from the implantation site without clogging the flow of the body fluid. Ultimately, the particles are eliminated from the body either by excretion in perspiration, urine or feces, or dissolved, degraded, corroded or otherwise metabolized into soluble components that are then excreted from the body.

A bioresorbable polymer is a resorbable polymeric material that is biocompatible. Preferred bioresorbable polymers are selected from polysaccharides, polyglycolic acid, polylactic acid, polycaprolactone, poly(ethylene terephthalate), poly(butic acid), poly(valeric acid), polyanhydrides, and polyorthoesters and blends and copolymers thereof. A bio compatible polymer is a polymeric material that is compatible with living tissue or a living system, non-toxic or non-injurious and do not cause immunological reaction or rejection. Preferred biocompatible polymers for use in the present invention include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonates), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

A preferred device according to the invention is a stent, preferably a stent comprising a generally tubular structure. A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

A preferred stent is a stent for treating narrowing or obstruction of a body passageway in a human or animal in need thereof. “Body passageway” as used herein refers to any of number of passageways, tubes, pipes, tracts, canals, sinuses or conduits which have an inner lumen and allow the flow of materials within the body. Representative examples of body passageways include arteries and veins, lacrimal ducts, the trachea, bronchi, bronchiole, nasal passages (including the sinuses) and other airways, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina and other passageways of the female reproductive tract, the vasdeferens and other passageways of the male reproductive tract, and the ventricular system (cerebrospinal fluid) of the brain and the spinal cord. Preferred devices of the invention are for these above-mentioned body passageways. Particularly preferred stents are however vascular stents. There is a multiplicity of different vascular stents known in the art per se that may be utilized following percutaneous transluminal coronary angioplasty.

Any number of stents may be utilized in accordance with the present invention and the invention is not limited to the specific stents that are described in exemplary embodiments of the present invention. The skilled artisan will recognize that any number of stents may be utilized in connection with the present invention. In addition, as stated above, other medical devices may be utilized, such as e.g. orthopedic implants.

The composition for controlled release comprised in a device according to the invention preferably comprises a second or further therapeutic agent in addition to the flavonoid as defined above. The second or further therapeutic agent may a therapeutic agent is selected from antiproliferative, antimitotic, antimicrobial, anticoagulant, fibrinolytic, anti-inflammatory, immunosurpressive, and anti-angiogenic agents. Examples of such second or further therapeutic and pharmaceutic agents for controlled release from the device include: cell cycle inhibitors in general, apoptosis-inducing agents, antiproliferative/antimitotic agents including natural products such as vinca alkaloids (i.e. vinblastine, vincristine, and vinorelbine), paclitaxel, colchicine, epidipodophyllotoxins (i.e. etoposide, teniposide), antibiotics (dactinomycin, actinomycin D, daunorubicin, doxorubicin, idarubicin, penicillins, cephalosporins, quinolones, etc.), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II.sub.b/III.sub.a inhibitors, GP-IIa inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine (cladribine)); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors (matrix protease inhibitors).

Preferably, the further therapeutic agent is an agent for preventing or reducing restenosis, more preferably for preventing or reducing restenosis subsequent to or associated with angioplasty. The further therapeutic agent preferably exhibits synergy with the flavonoid as defined above in preventing or reducing restenosis as well as in preventing or reducing secondary complications after angioplasty, including e.g. acute, subacute and chronic secondary complications associated with angioplasty such as thrombus, inflammation, and responses of the immunesystem. Suitable second or further therapeutic agents that exhibit synergy with the flavonoid as defined above include agents that are useful for treating restenosis and include known anti-inflammatory, anti-thrombogenic, anti-angiogenic, matrix protease inhibitory, anti-migratory, anti-proliferative (preferably a tubulin-binding anti-proliferative agent), cytostatic, and/or cytotoxic agents. Preferred agents are those that are currently being used or considered as stent coating materials to combat restenosis, which include paclitaxel, derivatives of paclitaxel, and sirolimus, a derivative of sirolimus. Particularly preferred are paclitaxel, sirolimus, tacrolimus and everolimus.

Analogues or derivatives of paclitaxel include docetaxel, BMS-184476, BMS 275183, BAY 59-8862, orataxel, taxumairol, taxinine M, taxacin, various baccatines and others described by Ya-Ching Shen et al., 2000, J. Chin. Chem. Soc., 47: 1125-30; Plummer et al., 2002, Clin Cancer Res. 8:2788-97; Agarwal et al., 2003, Curr. Oncol. Rep. 5: 89-98; and Jordan and Wilson, 2004, Nature Rev. Cancer 4: 253-65. Analogues of sirolimus (rapamycin) include C-7 Rapalog, AP 22594, 28-epi-rapamycin, 24,30-tetrahydro-rapamycin, AP 23573, trans-3-aza-bicyclo[3.1.0]hexane-2-carboxylic acid Rapamycin, ABT-578, SDZ RAD, CCI-779, AP 20840, AP 23464.

The dosage or concentration of e.g. paclitaxel based on surface area on a typical coronary stent may be is 0.1 and 5 μg/mm², preferably more than about 0.7 μg/mm² (at lower dosage restenosis rates are higher) and preferably less than about 3.0 μg/mm² (higher will be cytotoxic), more preferably between 1.0 and 1.8 μg/mm², and most preferably about 1.4 μg/mm². Consequently, the amount of paclitaxel will increase linearly with the length of the stent. E.g. for a typical series of coronary stent varying in length from 8.00 to 39.00 mm, the total paclitaxel content will vary from 50 μg to 250 μg. Suitable dosaging for drug-eluting stents is further described in U.S. Pat. No. 6,908,622.

The dosage or concentration of e.g. sirolimus based on surface area on a typical coronary stent may be is 0.1 and 5 μg/mm², preferably more than about 0.7 μg/mm² (at lower dosage restenosis rates are higher) and preferably less than about 3.0 μg/mm² (higher will be cytotoxic), more preferably between 1.0 and 1.8 μg/mm², and most preferably about 1.4 μg/mm² Consequently, the amount of sirolimus will increase linearly with the length of the stent. E.g. for a typical series of coronary stent varying in length from 8.00 to 39.00 mm, the total sirolimus content will vary from 50 μg to 250 μg.

Similarly the on-stent dosage may be determined for other therapeutic agents (including the flavonoids or derivatives thereof) by means known in the art. Usually, the amount of therapeutic agents will be dependent upon the particular drug employed and medical condition being treated. Typically, the amount of drug represents about 0.001 percent to about seventy percent of the total coating weight, more typically about 0.01 percent to about sixty percent of the total coating weight. Preferably, the weight percent of the therapeutic agents in the carrier or polymer coating is 1% to 50%, 2% to 45, 5% to 40, or 10 to 35%. It is however possible that the drug may represent as little as 0.0001 percent to the total coating weight.

A further preferred device according to the invention comprises at least two different compositions for controlled release of the flavonoid or derivative thereof as defined above and optionally the further therapeutic agent. Preferably the different compositions are present as subsequent layers at the surface of the device. The surface of the device is herein understood to mean any part of the device that, upon implantation of the device, comes in to direct contact with body fluids (as defined above) and/or (luminal) walls of the body passageway (as defined above). Preferably the entire surface of the device is coated/covered with one or more of the different compositions for controlled release although devices with only part of their surface coated with the composition are explicitly included in the invention. The different compositions may each differ from each other with respect to the concentration of at least one of the further therapeutic agents and the flavonoid or derivative thereof as defined above. Alternatively, the different compositions may each differ from each other with respect to the polymer-composition and/or concentration. A concentration of 0.0% (w/v or w/w), i.e. absence of a specific active agents or polymer is hereby included.

A particularly preferred device according to the invention is a device wherein the different compositions comprise a first composition comprising a flavonoid or derivative thereof as defined above and a second composition comprising paclitaxel or a derivative of paclitaxel and a flavonoid or derivative thereof as defined above. Optionally this device may comprise a third composition comprising paclitaxel or a derivative of paclitaxel and a flavonoid or derivative thereof as defined above whereby at least one of the concentrations of the active agents differ from the second composition. Examples of typical concentrations for the active agents and/or polymer compositions that may be applied on the devices are provided in Table 1.

Another particularly preferred device according to the invention is a device wherein the different compositions comprise a first composition comprising a flavonoid or derivative thereof as defined above and a second composition comprising sirolimus or a derivative of sirolimus and a flavonoid or derivative thereof as defined above. Examples of the typical concentrations for the active agents and/or polymer compositions that may be applied on the devices are provided in Table 2.

A device according to the invention further preferably comprises an external protective coating, whereby preferably the protective coating comprises no therapeutic agent. The purpose of the protective coating is to protect the various composition for controlled release from a variety of negative influences before and/or during implantation of the device. These influences include exposure to air and/or light which may degrade they active agents and/or polymers e.g. by oxidation, as well as to prevent the release of active ingredients during implantation of the device, before the devices reaches the site of implantation and already comes into direct contact with body fluids. The protective coating preferably is biocompatible or more preferably bioresorbable (as defined above) and will usually comprise a soluble polymer. The composition and thickness of the protective coating is preferably chosen such that the coating will have completely dissolved in a period between 30 minutes and several hours, in order not to unnecessarily delay the controlled release of the active agents. A suitable protective coating comprises polyvinyl pyrolidon (see also Tables 1 and 2).

The various compositions are numbered herein in the order that they are preferably applied only as examples. The first composition is thus first applied to the device and the second and further compositions are subsequently applied over the preceding composition. The composition with the highest number will thus be the first that dissolve upon implantation of the device, subsequently followed by the composition with the next lower number until the first composition is reached. However, the various different compositions may be applied to the device in any particular order to tailor the specific therapeutic requirements for a given condition. In a preferred embodiment, the first composition comprises only genistein as active agent (besides the polymer and optional exipients) such that it may be released from the device sequentially and in a controlled manner after the release of the second and further therapeutic agents (such as paclitaxel, rapamycin, and/or sirolimus or their derivates), to prevent the acute, subacute and chronic secondary complications of angioplasty.

The devices of the invention may be coated with the above defined compositions in a variety of manners. E.g. the composition(s) may be directly affixed to the device by either (air brush) spraying the device with a polymer/drug film, or by dipping the device into a polymer/drug solution; by coating the device stent with a first substance (such as a hydrogel) which is capable of absorbing the composition; or by constructing the device itself with a polymer/drug composition.

In a further aspect the invention relates to method for treating narrowing or obstruction of a body passageway the method comprising placing a medical device as defined in herein above in the narrowed or obstructed body passageway in a subject in need thereof. Thus methods are provided for expanding the lumen of a body passageway, comprising inserting a stent into the passageway, the stent having a generally tubular structure, the surface of the structure being coated with a composition comprising as defined above, such that the passageway is expanded. In the method, the body passageway may be selected from arteries, veins, lacrimal ducts, trachea, bronchi, bronchiole, nasal passages, sinuses, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina, the vasdeferens, and the ventricular system.

Generally, stents are inserted in a similar fashion regardless of the site or the disease being treated. Briefly, a preinsertion examination, usually a diagnostic imaging procedure, endoscopy, or direct visualization at the time of surgery, is generally first performed in order to determine the appropriate positioning for stent insertion. A guidewire is then advanced through the lesion or proposed site of insertion, and over this is passed a delivery catheter which allows a stent in its collapsed form to be inserted. Typically, stents are capable of being compressed, so that they can be inserted through tiny cavities via small catheters, and then expanded to a larger diameter once they are at the desired location. Once expanded, the stent physically forces the walls of the passageway apart and holds them open. As such they are capable of insertion via a small opening, and yet are still able to hold open a large diameter cavity or passageway. The stent may be self-expanding, balloon expandable or expandable by a change in temperature. Stents are typically manoeuvred into place under radiologic or direct visual control, taking particular care to place the stent precisely across the narrowing in the organ being treated. The delivery catheter is then removed, leaving the stent standing on its own as a scaffold. A post insertion examination, usually an x-ray, is often utilized to confirm appropriate positioning.

Within a preferred embodiment of the invention, methods are provided for eliminating vascular obstructions, comprising inserting a device according to the invention in the form of vascular stent into a blood vessel, the stent having a generally tubular structure, the surface of the structure being coated with a composition as described above, such that the vascular obstruction is eliminated. Briefly, stents may be placed in a wide array of blood vessels, both arteries and veins, to prevent recurrent stenosis (restenosis) at e.g. a site of (failed) angioplasties, to treat narrowings that would likely fail if treated with angioplasty, and to treat post surgical narrowings (e.g., dialysis graft stenosis). Thus in one aspect the invention provides for a method for the prevention or treatment of restenosis, preferably the method is method for the prevention or treatment of restenosis subsequent to angioplasty. In a further embodiment the invention provides for a method to inhibit neointimal hyperplasia subsequent to angioplasty.

Representative examples of suitable sites to be treated in the methods of the invention include e.g. the iliac, renal, and coronary arteries, the superior vena cava, and in dialysis grafts. Within one embodiment, angiography is first performed in order to localize the site for placement of the stent. This is typically accomplished by injecting radiopaque contrast through a catheter inserted into an artery or vein as an x-ray is taken. A catheter may then be inserted either percutaneously or by surgery into the femoral artery, brachial artery, femoral vein, or brachial vein, and advanced into the appropriate blood vessel by steering it through the vascular system under fluoroscopic guidance. A stent may then be positioned across the vascular stenosis. A post insertion angiogram may also be utilized in order to confirm appropriate positioning.

Again another embodiment of the invention relates to a method for the prevention of acute, subacute and chronic secondary complications associated with angioplasty. Such secondary complications subsequent to and/or associated with angioplasty are defined herein above and include e.g. restenosis, neointima, neointimal hyperplasia, thrombosis and inflammation.

In a further aspect the invention relates to the use of a flavonoid or derivative thereof for the manufacture of a medicament for the prevention or treatment of restenosis. Preferably the flavonoid is a flavonoid as defined above or a derivative thereof as defined above. A more preferred flavonoid is selected from genistein, quercetin, rutin, narigenin, naringin and derivatives thereof.

In a preferred use wherein the medicament is for the prevention or treatment of restenosis subsequent to angioplasty, more preferably the medicament is for the inhibition of neointimal hyperplasia subsequent to angioplasty.

In another embodiment the medicament is used for the prevention of an acute, subacute and chronic secondary complication associated with angioplasty. Preferably the secondary complication includes thrombus.

Again another embodiment of the invention includes a use as defined above wherein the medicament comprises a further therapeutic agent as defined above. Preferably the further therapeutic agent is selected from antiproliferative, antimitotic, antimicrobial, anticoagulant, fibrinolytic, anti-inflammatory, immunosurpressive, and anti-angiogenic agents, more preferably the further therapeutic agent is paclitaxel, a derivative of paclitaxel, sirolimus, a derivative of sirolimus, rapamycin, a derivative of rapamycin.

In a most preferred embodiment of the uses as defined above, the medicament is administered by implanting a device as defined above.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

### Manufacturing of a Stent Eluting Paclitaxel and Genistein Stent Manufacturing Process

The stent is manufactured from surgical grade Stainless Steel 316 L tube. Tubes are first cut with Laser Machine according to programmed design. The cut stents are electropolished for surface smoothness. These stents are transferred to clean room where quality check is carried out and further proceed to coating room where they are coated with Paclitaxel. The coated stents are crimped on rapid exchange balloon catheters. The packed stents are sterilized with EtO. Quality check is carried out at each and every stage and non-conform stents are rejected.

### Coating Process

Coating process consists of making solutions of Paclitaxel and Genistein with different Polymers and coating in three layers+a protective top coating. The stent thus contains four layers, layers 1, 2, 3 and 4, by respectively spraying solutions A, B, C and D (see Table 1). Coating process is carried out using aseptic conditions under controlled environment and clean room conditions. The temperature and humidity are maintained below 23° C. and 60% Rh respectively in clean room. The process is as follows. Check and set the drug coating machine parameters as per the stent size. Check the spray gun angle, distance between spray gun tip and stent and alignment of machine. Clean the gun with dichloromethane (DCM) before starting the coating process. Hang the stent between two collate with the help of hooks. Take the Solution ‘A’ as per loading calculation. ‘A’ contains Genistein+Poly 1-Lactide+Poly Vinyl Pyrrolidone+Dichloro Methane. Start the power supply of the coating machine. Start the spraying of solution on stent with optimum flow rate with necessary Nitrogen pressure. Wait till the complete solution is sprayed. During coating, maintain the flow rate. Dry the coating layer/s for 10 minutes after each coat. Remove the stent from collate and keep it in the centrifuge tube. After completion of ‘A’ layer coating, measure and record weight of stent. Check the surface of the stent in microscope. Similarly, complete B, C and D layer coating. Drug coated stents are kept in air tight centrifuge tube and transfer to clean room for further process. Solution B contains Genistein+Paclitaxel+Poly 1-Lactide+50/50 Poly DL Lactide-co-Glycolide+Poly Vinyl Pyrrolidone+Dichloro Methane Solution C contains Genistein+Paclitaxel+70/30 Poly L Lactide-co-Caprolactone+50/50 Poly DL Lactide-co-Glycolide+Poly Vinyl Pyrrolidone+Dichloromethane. Solution D contains Poly Vinyl Pyrrolidone+Dichloromethane.

TABLE 1
Amount of Paclitaxel and Genistein Incorporated on an 8 mm stents

| Layer | Polymer(s) | Genistein | Paclitaxel | Drug/polymer ratio |
|---|---|---|---|---|
| 1 (A) | Poly 1-Lactide + | 40 μg | — | 26/74 |
| | PVP | | | |
| 2 (B) | Poly 1-Lactide + | 40 μg | 35 μg | 34/66 |
| | 50/50 Poly DL | | | |
| | Lactide-co- | | | |
| | Glycolide + PVP | | | |
| 3 (C) | 70/30 Poly L | 20 μg | 16 μg | 20/80 |
| | Lactide-co- | | | |
| | Caprolactone + | | | |
| | 50/50 Poly DL | | | |
| | Lactide-co- | | | |
| | Glycolide + PVP | | | |
| 4 (D) | PVP | — | — | 0/100 |

### Example 2

### Manufacturing of a Stent Eluting Sirolimus and Genistein

Stent are essentially made as described above in Example 1 except that the stent contains three layers, layers 1, 2 and 3, by respectively spraying solutions A, B and D (see Table 2). Solution A contains Genistein+Poly 1-Lactide+50/50 Poly DL Lactide-co-Glycolide+PVP. Solution B contains Genistein+Sirolimus+70/30 Poly L Lactide-co-Caprolactone+50/50 Poly DL Lactide-co-Glycolide+Poly Vinyl Pyrrolidone+Dichloromethane Solution D contains Poly Vinyl Pyrrolidone+Dichloromethane.

TABLE 2
Amount of Sirolimus and Genistein Incorporated on an 8 mm stents

| Layer | Polymer(s) | Genistein | Sirolimus | Drug/polymer ratio |
|---|---|---|---|---|
| 1 (A) | Poly 1-Lactide + | 40 μg | — | 20/80 |
| | 50/50 Poly DL | | | |
| | Lactide-co- | | | |
| | Glycolide + PVP | | | |
| 2 (B) | 70/30 Poly L | 40 μg | 50 μg | 40/60 |
| | Lactide-co- | | | |
| | Caprolactone + | | | |
| | 50/50 Poly DL | | | |
| | Lactide-co- | | | |
| | Glycolide + PVP | | | |
| 3 (D) | PVP | — | — | 0/100 |

1-39. (canceled)

40. An implantable medical device comprising a composition for controlled release of a flavonoid or a derivative thereof, wherein the flavonoid or derivative thereof is capable of inhibiting tyrosine kinase, DNA topoisomerase-II and/or platelet aggregation, and wherein the composition comprises a further therapeutic agent in addition to the flavonoid or derivative thereof.

41. A device according to claim 40, wherein the flavonoid or derivative thereof is a flavanone, a flavonol, an isoflavone or derivatives thereof.

42. A device according to claim 41, wherein the flavonoid or derivative thereof is selected from genistein, quercetin, rutin, narigenin, naringin and derivatives thereof.

43. A device according to claim 41, wherein the composition for controlled release comprises a soluble polymer.

44. A device according to claim 43, wherein the soluble polymer is a biocompatible or bioresorbable polymer.

45. A device according to claim 44, wherein the bioresorbable polymer is selected from polysaccharides, polyglycolic acid, polylactic acid, polycaprolactone, poly(ethylene terephthalate), poly(butic acid), poly(valeric acid), polyanhydrides, and polyorthoesters and blends and copolymers thereof.

46. A device according to claim 40, wherein the device is a stent, preferably a stent comprising generally tubular structure.

47. A device according to claim 46, wherein the stent is for a body passageway selected from arteries, veins, lacrimal ducts, trachea, bronchi, bronchiole, nasal passages, sinuses, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina, the vasdeferens, and the ventricular system.

48. A device according to claim 40, wherein the further therapeutic agent is selected from antiproliferative, antimitotic, antimicrobial, anticoagulant, fibrinolytic, anti-inflammatory, immunosurpressive, and anti-angiogenic agents.

49. A device according to claim 40, wherein the further therapeutic agent is for preventing or reducing restenosis.

50. A device according to claim 40, wherein the further therapeutic agent is paclitaxel, a derivative of paclintaxel, sirolimus, or a derivative of sirolimus.

51. A device according to claim 40, wherein the device comprises at least two different compositions for controlled release of the flavonoid or derivative thereof and optionally the further therapeutic agent

52. A device according to claim 51, wherein the different compositions are present as subsequent layers at the surface of the device.

53. A device according to claim 51, wherein the different compositions each differ from each other with respect to the concentration of at least one of the further therapeutic agent and the flavonoid or derivative thereof.

54. A device according to claim 51, wherein the different compositions differ from each other with respect to the polymer-composition.

55. A device according to claim 51, wherein the different compositions comprise a first composition comprising the flavonoid or derivative thereof and a second composition comprising paclitaxel or a derivative of paclitaxel and the flavonoid or derivative thereof.

56. A device according to claim 51, wherein the different compostions comprise a first composition comprising the flavonoid or derivative thereof and a second composition comprising sirolimus or a derivative of sirolimus and the flavonoid or derivative thereof.

57. A device according to claim 40, wherein the device comprises an external protective coating, whereby preferably the protective coating comprises no therapeutic agent.

58. A device according to claim 57, wherein the protective coating is polyvinyl pyrolidon.

59. A method for treating narrowing or obstruction of a body passageway, the method comprising placing a medical device as defined in claim 40 in the narrowed or obstructed body passageway in a subject in need thereof.

60. A method according to claim 59, wherein the body passageway is selected from arteries, veins, lacrimal ducts, trachea, bronchi, bronchiole, nasal passages, sinuses, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina, the vasdeferens, and the ventricular system.

61. A method according to claim 60, wherein the method is a method for the prevention or treatment of restenosis.

62. A method according to claim 61, wherein the method is method for the prevention or treatment of restenosis subsequent to angioplasty.

63. A method according to claim 62, wherein the method is a method to inhibit neointimal hyperplasia subsequent to angioplasty.

64. A method according to claim 60, wherein the method is a method for the prevention or reduction of acute, subacute and chronic secondary complications associated with angioplasty.

65. A method according to claim 64, wherein the secondary complications include thrombus.

66. Use of a flavonoid or derivate thereof for the manufacture of a medicament for the prevention or treatment of restenosis, wherein the flavonoid or derivative thereof is capable of inhibiting tyrosine kinase, DNA topoisomerase-II and/or platelet aggregation, and wherein the medicament comprises a further therapeutic agent in addition to the flavonoid or derivative thereof.

67. A use according to claim 66, wherein the flavonoid is a flavanone, a flavonol, an isoflavone or derivatives thereof.

68. A use according to claim 67, wherein the flavonoid or derivative thereof is selected from genistein, quercetin, rutin, narigenin, naringin and derivatives thereof.

69. A use according to claim 66, wherein the medicament is for the prevention or treatment of restenosis subsequent to angioplasty.

70. A use according to claim 66, wherein the medicament is for the inhibition of neointimal hyperplasia subsequent to angioplasty.

71. A use according to claim 66, wherein the medicament is for the prevention of an acute, subacute and chronic secondary complication associated with angioplasty.

72. A use according to claim 71, wherein the secondary complication includes thrombus.

73. A use according claim 66, wherein the medicament comprises a further therapeutic agent.

74. A use according to claim 66, wherein the further therapeutic agent is selected from antiproliferative, antimitotic, antimicrobial, anticoagulant, fibrinolytic, anti-inflammatory, immunosurpressive, and anti-angiogenic agents.

75. A use according to claim 66, wherein the further therapeutic agent is paclitaxel, a derivative of paclitaxel, sirolimus, a derivative or sirolimus, rapamycin, a derivative of rapamycin.

76. A use according to claim 66, wherein the medicament is administered by implanting an implantable medical device comprising a composition for controlled release of a flavonoid or a derivative thereof.
